(19) United States
(12) Patent Application Publication
(10) Pub. No.: US 2020/0352447 A1
(43) Pub. Date: Nov. 12, 2020

(54) APPARATUS AND METHOD FOR ACQUIRING INFORMATION

(71) Applicant: CANON KABUSHIKI KAISHA, Tokyo (JP)

(72) Inventor: Toshinobu Tokita, Yokohama-shi (JP)

(21) Appl. No.: 16/298,513

(22) PCT Filed: Oct. 25, 2017
(86) PCT No.: PCT/JP2017/038543

§ 371 Date: Mar. 11, 2019

(30) Foreign Application Priority Data
Oct. 31, 2016 (JP) 2016-213423

(51) Int. Cl. A61B 5/00; A61B 5/107

(52) U.S. Cl. CPC A61B 5/0095; A61B 5/7475; A61B 5/742; A61B 2562/0242; A61B 5/489; A61B 2562/0204; A61B 5/1075

(57) ABSTRACT

A photoacoustic image of an object varies in contrast between a shallow portion and a deep portion according to the irradiation position of the object with respect to an ultrasonic probe. The present disclosure provides an information acquisition apparatus in which the contrast is high regardless of the depth of the region of interest. The information acquisition apparatus includes a varying unit that varies the irradiation position of the object with respect to the ultrasonic probe and controls the irradiation position according to an instruction on a condition for acquiring information on the object.

### TECHNICAL FIELD

The present disclosure relates to an information acquisition apparatus and a method for the same in which ultrasonic waves generated from an object is imaged by irradiating the object with illumination light.

### BACKGROUND ART

Photoacoustic imaging (PAI) is drawing attention as a method for specifically imaging angiogenesis caused by cancer. PAI is a method of applying illumination light (near infrared rays) to an object and receiving photoacoustic waves generated from the interior of the object with an ultrasonic probe to generate an image. FIG. 8 is a schematic diagram of a hand-held photoacoustic imaging apparatus disclosed in NPL 1.

In FIG. 8, an ultrasonic probe 801 is used to receive a photoacoustic signal. The photoacoustic signal received by the ultrasonic probe 801 is processed by a processing unit (not shown) to generate an image. A fiber 803 is used to transmit light emitted from a light source (not shown) to apply the illumination light to the object. An angle adjusting mechanism 808 is used to adjust the irradiation angle of the illumination light to the object and changes the angle of the fiber 803 with respect to the surface of the object. NPL 1 evaluates the intensity of the photoacoustic signal with respect to the depth of the object and the irradiation angle of the illumination light. The evaluation reveals that setting the depth of the object from 10 mm to 25 mm and the incidence angle of the illumination light between 40° and 50° provides a high-luminance photoacoustic signal.

### CITATION LIST

### Non Patent Literature

[NPL 1]
Christoph Haisch et al., Anal Bioanal Chem (2010) 397:1503-1510

### SUMMARY OF INVENTION

However, the related art has the following problems.

NPL 1 evaluates the intensity (luminance) of the photoacoustic signal with respect to the depth of the object and the incidence angle of the illumination light. However, it is practically necessary to evaluate the ratio of the intensity of the photoacoustic signal to the noise or the artifact at the time of imaging, that is, the degree of contrast. The inventor has found that the contrast is greatly influenced by the irradiation position with respect to the ultrasonic probe rather than the irradiation angle of the illumination light. In other words, the intensity of the photoacoustic signal increases, but the artifact increases, as the distance between the irradiation position of the illumination light and the ultrasonic probe decreases. In contrast, the intensity of the photoacoustic signal decreases, but the artifact also decreases, as the distance between the irradiation position of the illumination light and the ultrasonic probe increases. The ratio of the intensity of the photoacoustic signal to the artifact, that is, the contrast, also changes according the depth. It is therefore important to adjust the incidence position of the illumination light according to the depth of the object where the photoacoustic signal is acquired.

The present disclosure is made in consideration of the above problems.

The present disclosure improves the ratio of the intensity of a photoacoustic signal to artifacts, that is, the contrast.

### Solution to Problem

An information acquisition apparatus according to a first aspect of the present disclosure includes a light source, an ultrasonic probe, an information acquisition unit, a receiving unit, a varying unit, and a control unit. The light source is configured to apply light to an object. The ultrasonic probe is configured to receive a photoacoustic wave generated from the object irradiated with the light and convert the photoacoustic wave to an electrical signal. The information acquisition unit is configured to acquire information on the object based on the electrical signal. The receiving unit is configured to receive an instruction on a condition for acquiring the information on the object. The varying unit is configured to vary an irradiation position of the light applied from the light source to the object. The control unit is configured to control the varying unit. The control unit is configured to be capable of controlling the varying unit based on the instruction received by the receiving unit.

A method for acquiring information according to a second aspect of the present disclosure includes the step of receiving a photoacoustic wave generated from an object irradiated with light and converting the photoacoustic wave to an electrical signal, the step of acquiring information on the object based on the electrical signal, the step of receiving an instruction on a condition for acquiring the information on the object, and the step of controlling an irradiation position of the light applied to the object according to the instruction.

Further features of the present disclosure will become apparent from the following description of exemplary embodiments with reference to the attached drawings.

### BRIEF DESCRIPTION OF DRAWINGS

FIG. 1 is a diagram illustrating the overall configuration of a photoacoustic imaging apparatus according to an embodiment of the present disclosure.

FIG. 2A is a diagram illustrating a varying unit according to an embodiment of the present disclosure.

FIG. 2B is a diagram illustrating a varying unit according to another embodiment of the present disclosure.

FIG. 2C is a diagram illustrating a varying unit according to a still another embodiment of the present disclosure.

FIG. 2D is a diagram illustrating a varying unit according to a still another embodiment of the present disclosure.

FIG. 2E is a diagram illustrating a varying unit according to a still another embodiment of the present disclosure.

FIG. 2F is a diagram illustrating a varying unit according to a still another embodiment of the present disclosure.

FIG. 2G is a diagram illustrating a varying unit according to a still another embodiment of the present disclosure.

FIG. 2H is a diagram illustrating a varying unit according to a still another embodiment of the present disclosure.

FIG. 3A is a perspective view of a photoacoustic probe according to an embodiment of the present disclosure viewed from the object side.

FIG. 3B is a diagram illustrating a bowl-type ultrasonic probe.

FIG. 3C is an external view of the photoacoustic probe in FIG. 3A.

FIG. 4A is a flowchart for irradiation-position variable control according to a first embodiment of the present disclosure.

FIG. 4B is a graph showing the relationship between the depth of the RIO and the irradiation position.

FIG. 4C is a diagram illustrating irradiation positions.

FIG. 4D is a diagram illustrating irradiation-position variable control according to the first embodiment.

FIG. 5 is a diagram illustrating correction of light distribution according to a second embodiment of the present disclosure.

FIG. 6A is a diagram illustrating control of a total light amount according to a third embodiment of the present disclosure.

FIG. 6B is a graph showing a total light amount within the movable range of an irradiation position.

FIG. 7A is a diagram illustrating condition-setting for setting an irradiation position according to a fourth embodiment of the present disclosure.

FIG. 7B is a flowchart for condition-setting image acquisition.

FIG. 7C is a schematic diagram illustrating part of a photoacoustic image.

FIG. 8 is a schematic diagram of the configuration of a photoacoustic imaging apparatus of a related art.

### DESCRIPTION OF EMBODIMENTS

A varying unit that varies the irradiation position to an object with respect to an ultrasonic probe is provided, and the irradiation position is controlled according to the region of interest of the operator.

The following description is intended to refer to specific embodiments of the present disclosure and is not intended to limit the present disclosure.

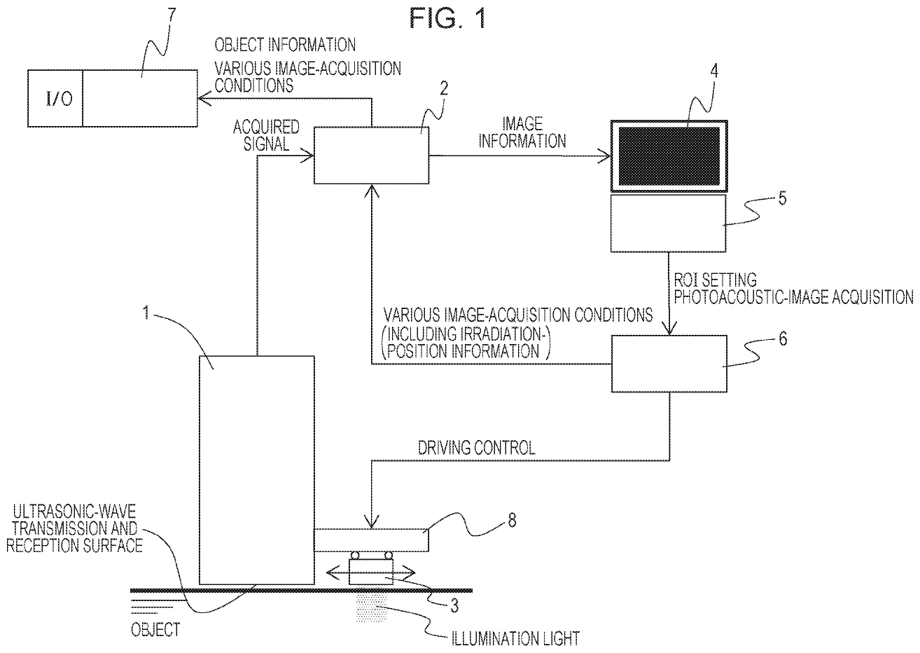

FIG. 1 schematically illustrates a photoacoustic imaging (PAI) apparatus serving as an information acquisition apparatus. In FIG. 1, an ultrasonic probe 1 is used to receive acoustic waves and convert the acoustic waves to an electrical signal. The ultrasonic probe 1 can also receive ultrasonic waves to an object and receive the ultrasonic waves reflected from the interior of the object. The ultrasonic-wave transmitting and receiving surface of the ultrasonic probe 1 is acoustically in contact with the object via an acoustic matching agent (sonar gel, water, etc.) (not shown).

An information processing unit (information acquisition unit) 2 is used to generate an image by amplification, analog-to-digital conversion, and filtering of the photoacoustic signal or the ultrasonic signal received by the ultrasonic probe 1. The information processing unit 2 is capable of beam forming when transmitting or receiving ultrasonic waves with the ultrasonic probe 1. A light source 3 is used to emit illumination light toward the object.

The light source 3 is a solid-state laser using neodymium-doped yttrium aluminum garnet (Nd:YAG), titanium sapphire (Ti:sa), optical parametric oscillation (OPO), or alexandrite. The light is transmitted to an emission end of the light source 3 through a bundle fiber or the like (not shown). The light source 3 is not limited to the solid-state laser but may be a laser diode (LD) or a light-emitting diode (LED). The light may not be transmitted through the bundle fiber. The light source 3 needs to emit pulsed light of several nanoseconds to a few hundred nanoseconds to generate a photoacoustic signal. The pulsed light may be rectangular or Gaussian in shape.

A monitor (display unit) 4 is configured to be capable of display information on the object, generated by the information processing unit (information acquisition unit) 2, typically, image information on the object. The monitor 4 includes a display control unit configured to be capable of controlling display of the image information on the object.

A receiving unit (input unit) 5 is used to receive an instruction concerning conditions for acquiring object information, that is, an instruction concerning an image-acquisition condition for acquiring a photoacoustic or ultrasonic image and to set the conditions. An example of the conditions for acquiring the information on the object is information on the region of interest of the object, such as the depth of the region of interest of the object. Another example is an irradiation position. The receiving unit 5 of the present embodiment includes an input unit to allow inputting instructions concerning the conditions for acquiring information on the object. Examples of the input unit of the present embodiment include pointing devices, such as a mouse, a trackball, and a touch panel.

A control unit 6 is used to perform various control operations on the basis of image acquisition conditions input with the input unit. The information on the image acquisition conditions is sent from the control unit 6 to the information processing unit 2 and is reflected to the processing operation of the information processing unit 2. For example, when acquisition of a photoacoustic image is started by the operation of the input unit 5, the information processing unit 2 stops transmission of ultrasonic waves and causes the light source 3 to emit illumination light. For acquisition of an ultrasonic image, selection of an image acquisition mode, such as B-mode tomography, color Doppler, or power Doppler, and focus setting in the object are operated with the input unit 5. The information processing unit 2 performs beam forming according to the operation to cause the ultrasonic probe 1 to transmit and receive ultrasonic waves to form an image.

A recording unit 7 is used to record the object information and various image acquisition conditions generated by the information processing unit 2. Furthermore, the object information and the various image acquisition conditions can be transferred to a computer in a medical facility over a network or to an external storage device (not shown), such as a memory or a hard disk, from the recording unit 7 via an I/O.

In the above photoacoustic imaging apparatus, a varying unit (an irradiation position varying unit) 8 is used to vary the lighting position (irradiation position) of light by driving the emission end of the light source 3 with respect to the ultrasonic probe 1. The varying unit 8 includes, for example, an actuator that varies at least part of the emission end of light emitted from the light source 3.

In acquiring a photoacoustic image of the object, a region of interest (ROI) in the object is set with the input unit 5. This may be paraphrased as focus position setting in ultrasonic image acquisition. This allows the light source 3 (emission end 301) to be brought closer to or away from the ultrasonic probe 1 according to the ROI setting.

For example, if the ROI is a shallow region of the object, the varying unit 8 bring the light source 3 (emission end) close to the ultrasonic probe 1. This is effective for acquiring an image of the skin and subcutaneous vessels in a relatively shallow portion of the object. By applying the illumination light to a portion close to the image acquisition target, a high-contrast image is acquired. If the ROI is a deep region, the varying unit 8 brings the light source 3 (emission end 301) away from the ultrasonic probe 1. This is effective for acquiring an image of deep inflammatory vessels and tumor vessels. This prevents application of strong illumination light to an object in the vicinity of the ultrasonic-wave transmission and reception surface of the ultrasonic probe 1. This suppresses photoacoustic waves generated from tissue with high light absorption, such as a skin and subcutaneous vessels below the transmission and reception surface, reducing noise and artifacts to generate a high-contrast image.

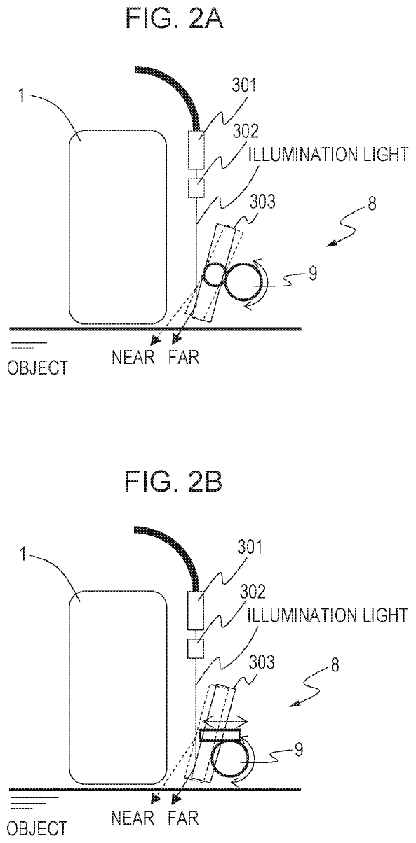

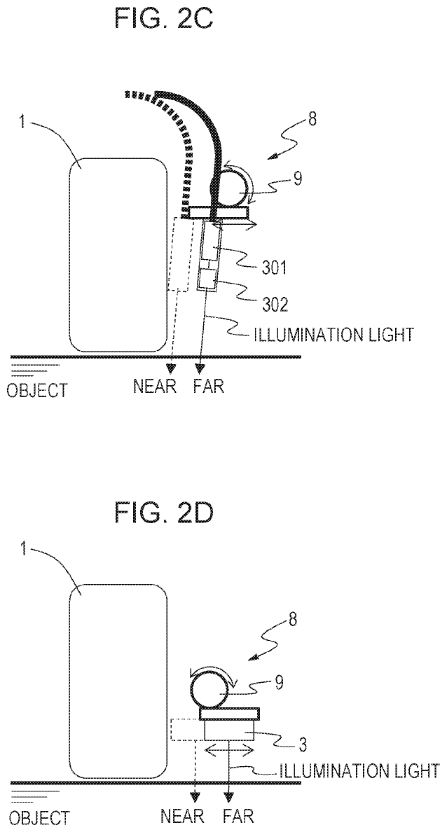

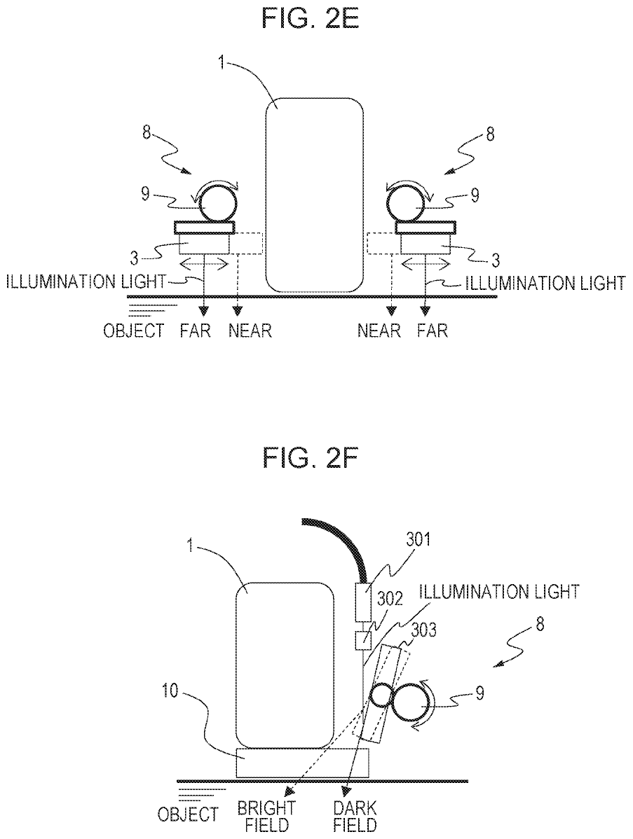

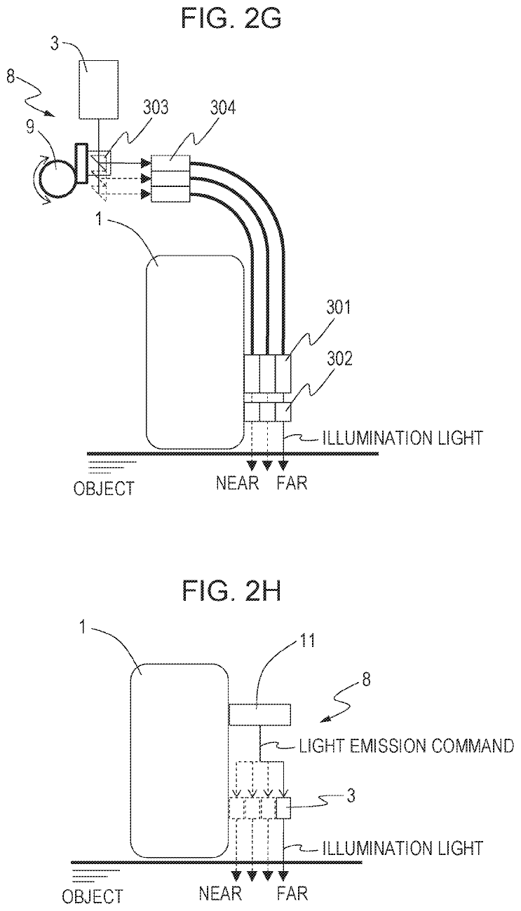

Next, the varying unit 8 will be described with reference to FIGS. 2A to 2H. FIG. 2A illustrates a configuration in which the irradiation position can be largely moved using a rotation mechanism including an actuator. The illumination light generated from the light source 3 (not shown) is transmitted through a fiber and is emitted from an emission end 301 of the fiber. Since the light emitted from the fiber spreads, the light may be formed using an optical element 302. Examples of the optical element 302 include a lens and a diffuser. The illumination light is bent by a reflective element 303 onto object. An actuator 9 is used to change the angle of the reflective element 303 by driving. This allows the illumination light to be varied in irradiation position from a near irradiation position to a far irradiation position with respect to the ultrasonic probe 1.

Although the varying unit 8 in FIG. 2A uses a rotation mechanism, the reflective element 303 may be translated using the actuator 9 and a rack and pinion mechanism, as illustrated in FIG. 2B.

Alternatively, as illustrated in FIG. 2C, an emission end portion from the emission end 301 of the fiber to the optical element 302 may be moved together using the actuator 9.

In FIG. 2D, the light source 3 is disposed in the vicinity of the ultrasonic probe 1 without using the fiber. In this case, a solid-state laser, such as a Nd:YAG laser, is difficult to place, so that the light source 3 may be a compact light-emitting device, such as a LD or a LED. This eliminates the need for routing a fiber, reducing the size and improving the usability.

FIGS. 2A to 2D illustrate a configuration in which light is applied to the object from one side of the ultrasonic probe 1. Alternatively, light may be applied from both sides of the ultrasonic probe 1, as illustrated in FIG. 2E. Furthermore, the distances of the irradiation positions from the ultrasonic probe 1 may differ from each other.

The configurations in FIGS. 2A to 2E are such that the irradiation position(s) can be varied at a position(s) away from a portion(s) of the object facing the ultrasonic probe 1, that is, under dark-field illumination, but this is not intended to limit the present disclosure. Referring to FIG. 2F, an acoustic matching material 10 made of urethane resin, polymethylpentene, or resin containing water as a main component is disposed. The acoustic matching material 10 that allows light and photoacoustic waves to pass through is disposed between the ultrasonic probe 1 and the object, so that the illumination light is applied to the object through the acoustic matching material 10. This allows the illumination light to be applied to a portion of the object facing the ultrasonic probe 1. In other words, this allows bright-field illumination. This allows switching between the bright-field illumination and the dark-field illumination, or setting to an irradiation position between them. The bright-field illumination means that the light irradiation position is set to a bright-field region using the varying unit 8, and the dark-field illumination means that the light irradiation position is set to a dark-field region using the varying unit 8.

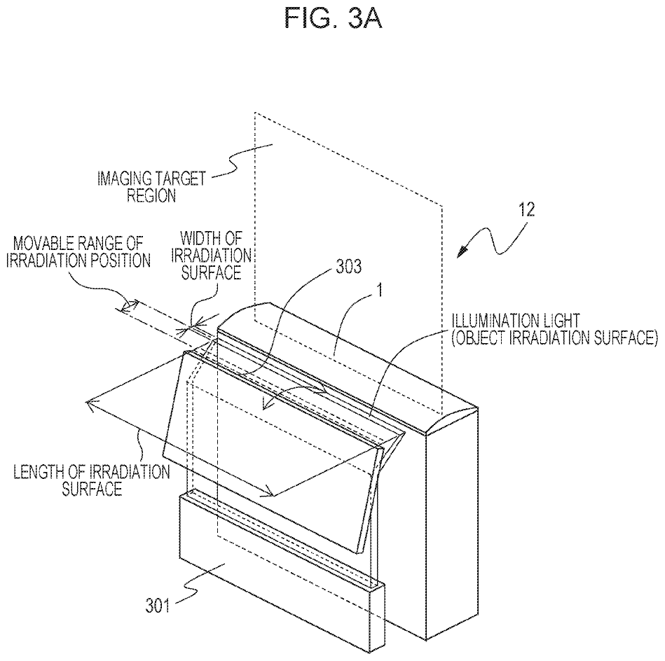

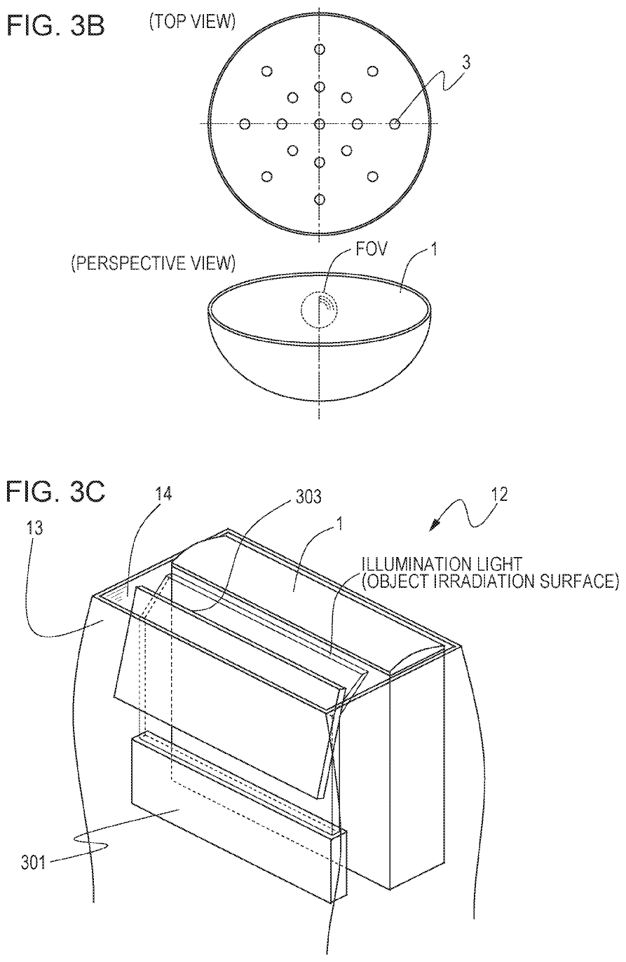

The bright-field illumination provides the strongest signals of a skin and subcutaneous tissue and the strongest contrast. This increases the depthwise image-acquisition range from the surface of the object, in other words, the skin and subcutaneous tissue, to a deep part of the object. An acoustic matching agent, such as sonar gel or water, is disposed between the acoustic matching material 10 and the object so that the ultrasonic probe 1 and the object are acoustically in contact with each other. Although the acoustic matching material 10 has been described as resin, the acoustic matching material 10 may be water or another liquid in the case where the ultrasonic probe 1 can be held with liquid, such as when used upward. For example, in the case of a bowl-shaped probe, as illustrated in FIG. 3B described later, the acoustic matching material 10 may be water. The acoustic matching material 10 needs to be a medium that transmits illumination light and ultrasonic waves.

Although the configurations in FIGS. 2A to 2F are such that the irradiation position(s) are varied by moving part or the whole of the emission end(s) 301 using the actuator 9, this is not intended to limit the present disclosure. The configuration in FIG. 2G is such that a plurality of emission ends 301 are provided, and the actuator 9 is driven at a position close to the light source 3 to move the reflective element 303 to switch illumination light incident on the incidence end 304 of the fiber. This configuration allows the irradiation position of the illumination light to be varied. This allows the actuator 9 to be disposed at a position separate from the ultrasonic probe 1, reducing the peripheral size of the ultrasonic probe 1.

Furthermore, the same number of light sources 3 as the number of the emission ends may be provided to allow switching among the light sources 3. In this case, using expensive solid-state lasers, such as Nd:YAG lasers, as the light sources 3 will increase the overall cost. For that reason, relatively inexpensive light-emitting devices, such as LDs or LEDs, may be used. Compact light sources 3, such as LDs or LEDs, can be disposed in the vicinity of the ultrasonic probe 1. Referring to FIG. 2H, a light-emission control unit 11 is used to control light emission from the light sources 3 and includes a light-emission driver. A plurality of light sources 3 or emission ends of the light source 3 are provided, and the light sources 3 or the emission ends of the light source 3 can be switched using the light-emission control unit 11 so that the irradiation position is variable. This eliminates the need for the actuator 9 and also the fiber, reducing the size and improving the usability.

Driving of the actuator 9 and switching of the light-emission control unit 11 are performed by the control unit 6 illustrated in FIG. 1. Although members for fixing the emission end(s) 301 and the light-mitting unit(s) and components that move with driving of the actuator 9 are not illustrated for the sake of simplifying the diagrams, these members and components are of course provided.

Next, a photoacoustic probe 12 including the ultrasonic probe 1 will be described with reference to FIGS. 3A to 3C.

FIG. 3A is a perspective view of the photoacoustic probe 12 viewed from the object side. Descriptions of the actuator 9 and so on illustrated in FIGS. 2A to 2H are omitted. Referring to FIG. 3A, the ultrasonic probe 1 is a one-dimensional (1D)-array linear probe. FIG. 3A illustrates an irradiation surface on which the illumination light illuminates the object and its movable range. The width and the longitudinal length of the irradiation surface are substantially the same as those of the ultrasonic probe 1. The width and the length can be changed so that the exposure amount is equal to or less than a maximum permissible exposure (MPE) to skin (JISC6802, ANSI Z136.1) according to the total amount of light applied. For example, the MPE when the total light amount is 20 mJ, the light emission frequency is 10 Hz, and the wavelength is 750 nm is about 25 mJ/cm². Therefore, when the ultrasonic probe 1 is 40 mm long, setting the length and the width of the irradiation surface respectively 35 mm and 2.5 mm can make the exposure amount equal to or less than the MPE. The movable range of the irradiation position is set to half or more (½ or more) of the width of the irradiation region of illumination light applied to the object, preferably, twice or more in a direction near to or far from the ultrasonic probe 1. In other words, the direction near to or far from the ultrasonic probe 1 is a direction near to or far from the imaging target region in the object.

The ultrasonic probe 1 is not limited to the 1D-array probe. Applicable examples include a probe that mechanically scan a 1D array, a two-dimensional (2D)-array probe, a sector type, a convex type, and a concave type. FIG. 3B illustrates a bowl-type ultrasonic probe 1. The ultrasonic probe 1 in FIG. 3B has receiving elements (not shown) arranged therein. The ultrasonic probe 1 includes a plurality of light sources 3 (emission ends 301). The ultrasonic probe 1 has an area called the field of view (FOV) in the vicinity of the center of curvature of the ultrasonic probe 1. The irradiation position is varied with respect to the FOV. For that purpose, the irradiation position is switched, as illustrated in FIGS. 2G and 2H.

FIG. 3C is an external view of the photoacoustic probe 12. In FIG. 3C, reference sign 13 denotes a casing. The casing 13 contains the ultrasonic probe 1 and the emission end 301 of the light applied to the object. Although the corners and ridges of the casing 13 are illustrated, actually the corners and the ridges may be tapered or rounded. In particular, for a hand-held type, the casing needs a curve or a hollow so that the operator can easily grip the casing. A cover (covering unit) 14 is used to protect a surface adjacent to the object. a thin resin, such as polyethylene terephthalate (PET) or urethane rubber, is bonded to the object-side surface of the casing 13. The cover 14 prevents the acoustic matching agent, such as sonar gel or water, from entering the casing, reducing or eliminating the trouble of the actuator 9 (not shown) in the photoacoustic probe 12. Although in FIG. 3C the cover 14 does not cover the receiving surface of the ultrasonic probe 1, the cover 1 may cover the receiving surface. Furthermore, the receiving surface of the ultrasonic probe 1 is coated with a light reflecting coating, such as chromium or gold. The light reflecting coating reduces or eliminates photoacoustic waves, which are generated when scattered illumination light is incident on the receiving surface of the ultrasonic probe 1, reducing noise sources, improving the contrast.

Embodiments will be described hereinbelow.

### [Method for Acquiring Information]

A method for acquiring information according to an embodiment of the present embodiment includes at least the following steps of: receiving a photoacoustic wave generated from an object irradiated with light and converting the photoacoustic wave to an electrical signal; acquiring information on the object based on the electrical signal; receiving an instruction on a condition for acquiring the information on the object; and controlling an irradiation position of the light applied to the object according to the instruction. The control step includes the step of changing the irradiation position of the light to the object according to the depth of a region of interest of the object when an instruction on the depth of the region of interest is received at the receiving step.

The method may further include the following steps of: performing condition-setting image acquisition for acquiring a photoacoustic image while controlling the irradiation position within a movable range of the irradiation position; displaying the irradiation position during the condition-setting image acquisition on a display unit; upon receiving an instruction on the irradiation position, controlling the irradiation position according to the instruction; and acquiring a photoacoustic image of the object at the irradiation position.

The method may further include the following steps of: upon setting a region of interest of the object and starting the condition-setting image acquisition, performing condition-setting image acquisition for acquiring the information on the object while controlling the irradiation position within a movable range of the irradiation position; determining the irradiation position where the set region of interest has high contrast; controlling the irradiation position according to the determination; and acquiring a photoacoustic image of the object at the irradiation position.

### EMBODIMENTS

### First Embodiment

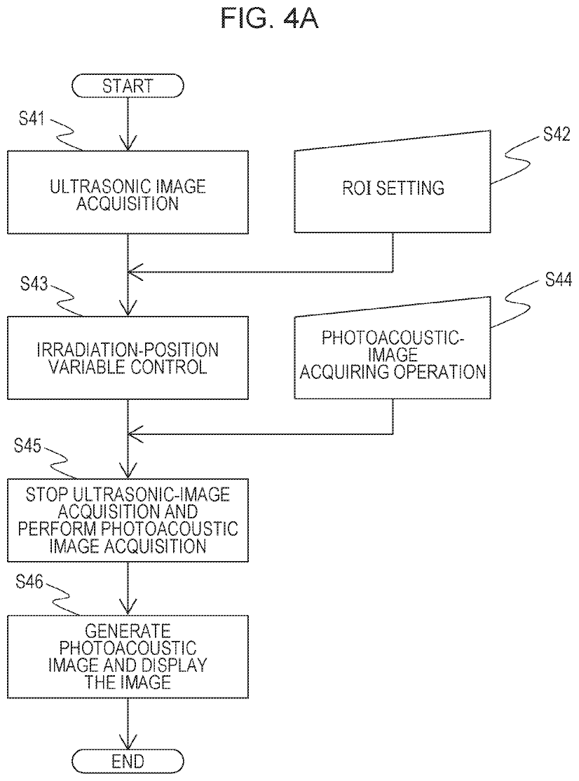

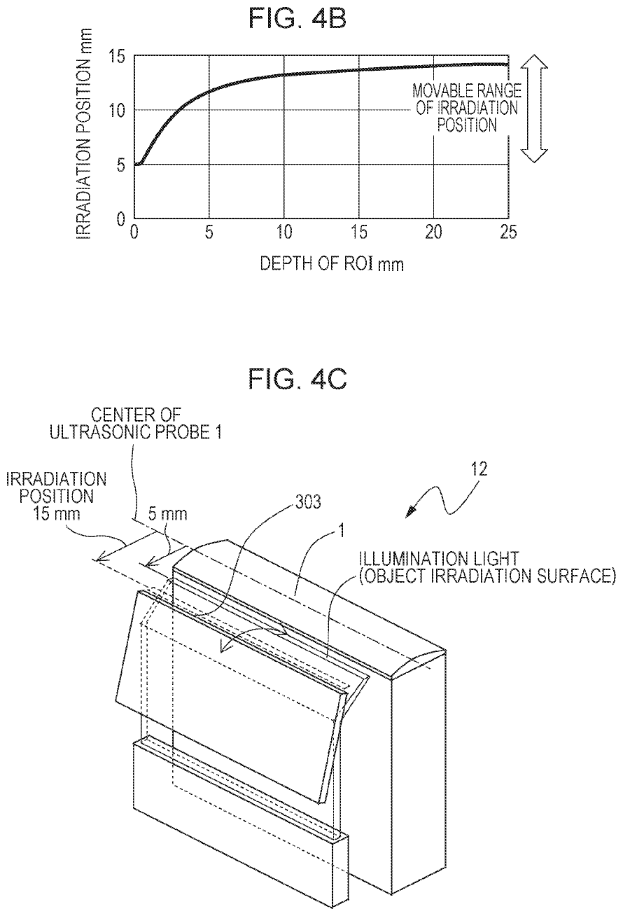

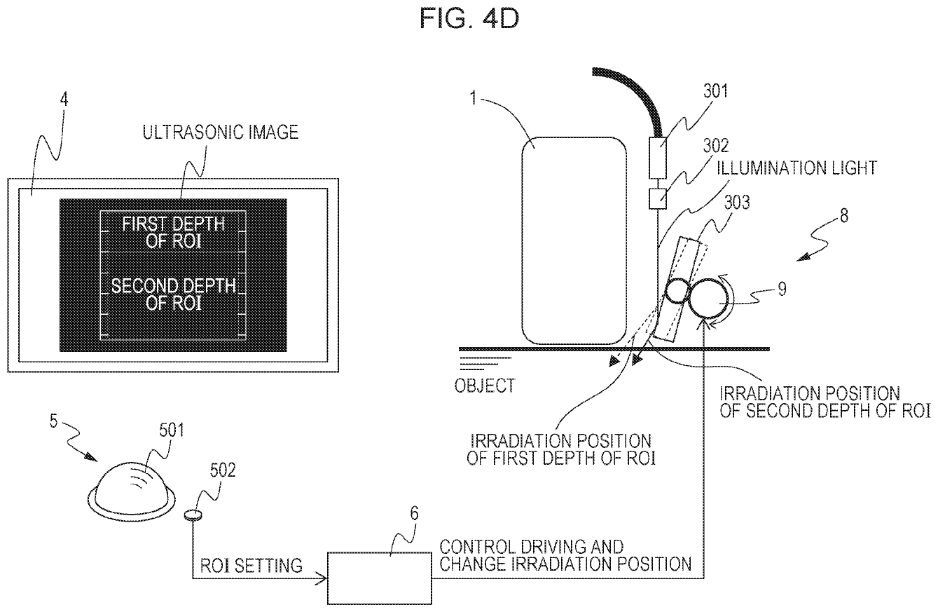

In a first embodiment, irradiation-position variable control will be described with reference to FIG. 1 and FIGS. 4A to 4D. Referring to FIG. 4A, the variable control includes the following steps.

Step 41 (S41) is an ultrasonic image acquisition step. A transmitted beam subjected beamforming by the information processing unit 2 is transmitted from the ultrasonic probe 1 into the object. Ultrasonic waves reflected from the interior of the object is received with the ultrasonic probe 1. The received signal is amplified, converted from analog to digital, and filtered by the information processing unit 2 to generate an ultrasonic image, and the ultrasonic image is displayed on the monitor 4.

Step 42 (S42) is a ROI setting step. The operator sets the region of interest with the input unit 5 while viewing the ultrasonic image displayed at S41.

Step 43 (S43) is an irradiation-position variable control step, at which the control unit 6 varies the irradiation position of the illumination light using the varying unit 8 according to the depth of the ROI set by the operator.

Step 44 (S44) is a photoacoustic-image acquisition step, at which the operator performs a photoacoustic-image acquiring operation with the input unit 5.

At step 45 (S45), the ultrasonic image acquisition is stopped according to the operation at S44, and photoacoustic image acquisition is performed. In the case where light emission and signal acquisition are performed a plurality of times to acquire a photoacoustic image, the ultrasonic image acquisition may be performed between the signal acquisition and the next light emission.

Step 46 (S46) is an imaging step, at which the photoacoustic signal received by the ultrasonic probe 1 is amplified, converted from analog to digital, and filtered by the information processing unit 2 to generate a photoacoustic image, and the photoacoustic image is displayed on the monitor 4. The monitor 4 displays the ultrasonic image acquired at S41 in monochrome in a superimposed manner and the photoacoustic image acquired at S46 in color in a superimposed manner. The monochrome and the color may be reversed, and the images may be displayed side by side without superimposing or may be displayed in a switched manner.

Next, the varying operation of the varying unit 8 at S43 will be described. The depth of the ROI of the object is determined from the center of the ROI set at S42. Although the center of the ROI is employed in defining the depth of the object, this is not limited thereto. The shallowest or the deepest portion may be employed for definition. The irradiation position of the illumination light is determined from the depth of the ROI. In the present embodiment, the irradiation position is determined as the distance from the ultrasonic probe 1 to an irradiation position C1+C2*exp (−C3/(depth of the RIO)), with the portion under the center of the ultrasonic probe 1 at zero. For the bright-field illumination, C1=0 is satisfied, while in the range of dark-field illumination, C1 is a position closest to the ultrasonic probe 1. FIG. 4B illustrates the relationship between the depth of the RIO and the irradiation position when C1=5, C2=10, and C3=2. FIG. 4C illustrates an irradiation position of 5 mm closest to the ultrasonic probe 1 (indicated by a thin solid line), and an irradiation position of 15 mm furthest from the ultrasonic probe 1 (indicated by a thin dashed line). These factors C1, C2, and C3 may also be adjusted depending on the region of the object and are not limited to the values. The movable range of the irradiation position has been described as 5 mm to 15 mm. However, the movable range is not limited to the values, which applies also to the following embodiments.

The distance from the ultrasonic probe 1 to the irradiation position is expressed as an exponential function. Alternatively, the distance may be expressed as an expression using a linear function or a higher-order function. Alternatively, a reference table in which the variable amounts of the varying unit 8 are listed according to the depth of the ROI. In other words, the control unit 6 can be configured to control the varying unit 8 on the basis of the expression or the table for determining the irradiation position according to the depth of the ROI of the object, which is received by the receiving unit (input unit) 5.

These methods allow the irradiation position to be determined upon setting of the depth of the RIO.

In the case where the table is referred to to determine the variable amount of the varying unit 8, the table includes at least two stages. In FIG. 4D, an ultrasonic image is displayed on the monitor 4. The operator sets the ROI on the ultrasonic image using the input unit 5. The input unit 5 includes a trackball 501 and an operating switch 502. When the ROI is set, the control unit 6 determines whether the RIO is a first depth of the RIO or a second depth of the RIO from the depth of the ROI. This can be determined from whether the ROI is set in the region of the first depth of the RIO or the region of the second depth of the RIO displayed on the monitor 4 in FIG. 4D. In this case, a depth of 10 mm from the surface of the object is defined as the first depth of the RIO (the region of interest is shallow), and a depth of 10 mm or more is defined as the second depth of the RIO (the region of interest is deep) but is not limited thereto. The irradiation position is changed according to the region of the depth of the RIO. Thus, the method of setting the irradiation position to a position corresponding to the depth of the designated ROI is also advantageous. Although the depth of the RIO in FIG. 4D has two stages, the number of stages may be increased. As described above, the control unit 6 is configured to perform control such that, when the region of interest is shallow, the irradiation position of light from the light source 3 to the object is brought close to the ultrasonic probe 1, and when the region of interest is deep, the irradiation position of light from the light source 3 to the object is brought away from the ultrasonic probe 1. The control unit 6 may also be configured to bring the irradiation position of light from the light source 3 to the object away from the ultrasonic probe 1 as the depth of the region of interest of the object increases.

Although the first embodiment has been described on the assumption that one illumination light is applied, two illumination lights may be applied from both sides of the ultrasonic probe 1, as illustrated in FIG. 2E. Furthermore, the irradiation positions may be symmetrical about the ultrasonic probe 1. When a plurality of depths of RIOs are present, the irradiation positions may be independently set.

### Second Embodiment

In a second embodiment, correction of light distribution and control of the total light amount according to the irradiation position will be individually described.

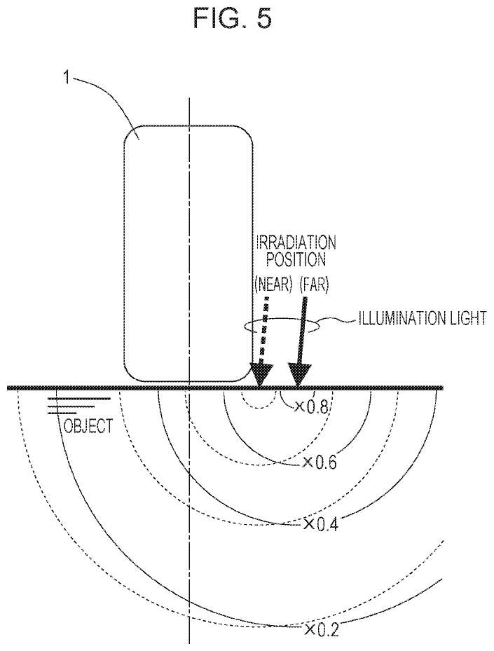

FIG. 5 schematically illustrates a light amount distribution in the object in a case where the irradiation position of the illumination light is close to the ultrasonic probe 1 (dashed line) and a case where the irradiation position is far from the ultrasonic probe 1 (solid line). The lines of ×0.8, ×0.6, ×0.4, ×0.2 in FIG. 5 indicate the magnification ratios of the amount of light absorbed and attenuated in the object to the total amount of the illumination light. FIG. 5 shows that the amount of light varies depending on whether the irradiation position is close to or far from the center line of the ultrasonic probe 1 at which the ultrasonic probe 1 obtains signals. In other words, the total amount of light applied to the object is controlled according to the irradiation position of light from the light source 3 to the object.

Thus, when the irradiation position changes, the light amount distribution in the object changes. The initial sound pressure p of the photoacoustic signal is expressed as Γ×μa×φ, where Γ is a Grueneisen constant, μa is an absorption coefficient, φ is light amount. The absorption coefficient μa is given by μa=p/(Γ×φ). The initial sound pressure p is obtained by converting a received sound pressure, and Grueneisen constant Γ is a known value. Therefore, the absorption coefficient μa can be calculated if the light amount φ is found out. Thus, the light amount distribution in the object is calculated by the information processing unit 2 on the basis of the irradiation position information set by the control unit 6 in FIG. 1. The light amount distribution can be calculated using a thermal diffusion equation or a Monte Carlo method from the optical constant μeff of the internal tissue of the object, the total light amount of the illumination light, the irradiation position, and the light amount distribution on the surface of the object. The light amount distribution can be calculated using the irradiation position as a parameter because the above factors other than the irradiation position are known values if measured in advance. This calculation does not have to be performed each time the irradiation position is changed. The light amount distribution in the object with respect to the irradiation positions may be written in a tabular form, or a conversion formula thereof may be provided.

The above configuration allows the light amount distribution in the object to be found out, improving the calculation accuracy of the absorption coefficient in the object. Furthermore, the above configuration also improves the calculation accuracy of oxygen saturation obtained from photoacoustic signals obtained while changing the wavelength of the illumination light. The information processing unit is configured to be able to calculate at least one of the light amount distribution in the object, the absorption coefficient in the object, and the oxygen saturation in the object. The information processing unit may be configured to be able to calculate the light amount distribution in the object, the absorption coefficient in the object, and the oxygen saturation in the object according to the irradiation position of light from the light source 3 to the object and the total light amount of light applied to the object.

### Third Embodiment

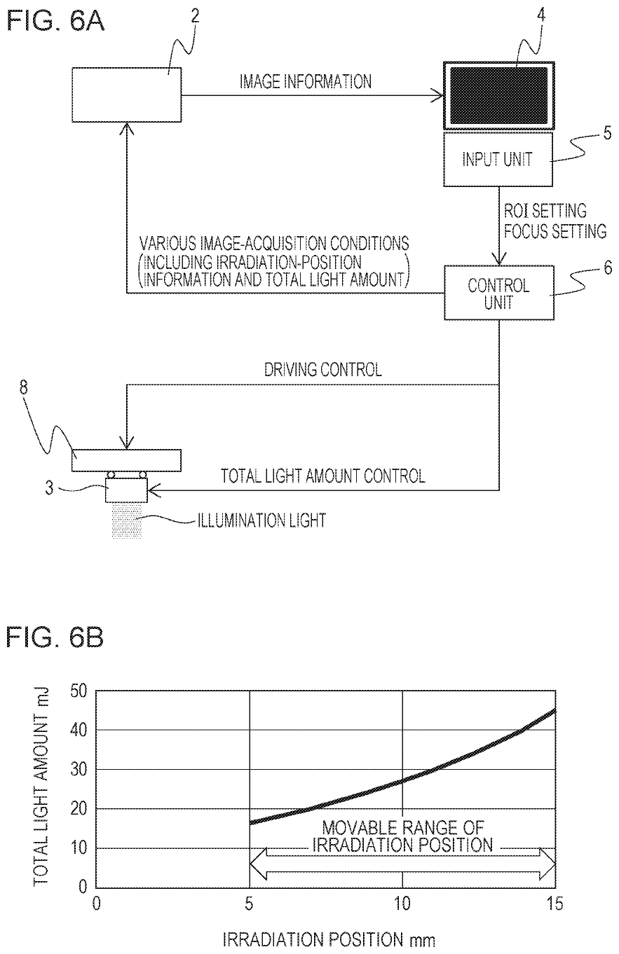

As described in the second embodiment with reference to FIG. 5, the light distribution of the object varies according to the irradiation position, that is, the light amount of the image acquisition region (the area indicated by the dashed line below the ultrasonic probe 1 in FIG. 5) decreases with an increasing distance between the irradiation position and the ultrasonic probe 1. For that reason, in a third embodiment, the total light amount is controlled according to the irradiation position. Referring to FIG. 6A, the control unit 6 not only controls the varying unit 8 to variably control the irradiation position of the illumination light but also the total amount of light to be emitted from the light source 3. The total light amount [mJ]=C4*exp (the distance [mm]/C5 from the ultrasonic probe 1 to the irradiation position), where C4=10, C5=10. The movable range of the irradiation position is set from 5 mm to 15 mm with reference to the center of the ultrasonic probe 1, and the total light amount within the range is shown in FIG. 6B. Thus, in the third embodiment, when the irradiation position is close to the ultrasonic probe 1, that is, the RIO is shallow, image acquisition is possible even if the light amount is small, and when the irradiation position is far from the ultrasonic probe 1, that is, the RIO is deep, image acquisition is possible at a large light amount. Since the total light amount can be controlled according to the irradiation position, the contrast of the image can be kept constant regardless of the depth of the RIO. The factors C4 and C5 are not limited to the above values. Not the exponential function but a linear function or a high-order function may be employed. The illumination light is formed so as to achieve the MPE or less to the skin even if the total light amount is increased.

Furthermore, this may be reflected to the calculation of the light amount distribution described in the second embodiment. The control unit 6 sends not only the irradiation position but also total light amount information to the information processing unit 2, and the information processing unit 2 calculates the light amount distribution in the object using the irradiation position and the total light amount as parameters. This allows the light amount distribution in the object to be calculated even if the light amount is varied as the irradiation position changes, improving the calculation accuracy of the absorption coefficient in the object and the calculation accuracy of the oxygen saturation.

### Fourth Embodiment

In the first embodiment, a method in which the operator sets the region of interest and varies the irradiation position with the varying unit 8 has been described. In a fourth embodiment, a condition setting method will be described. The method is such that the operator sets an irradiation position at which a desired photoacoustic image is acquired while moving the irradiation position in a variable range.

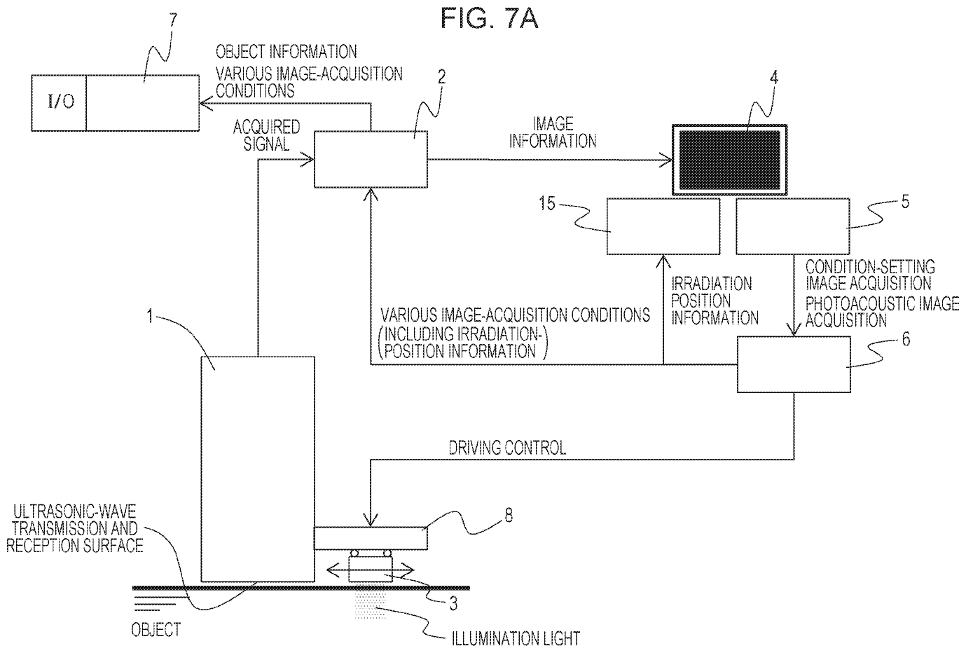

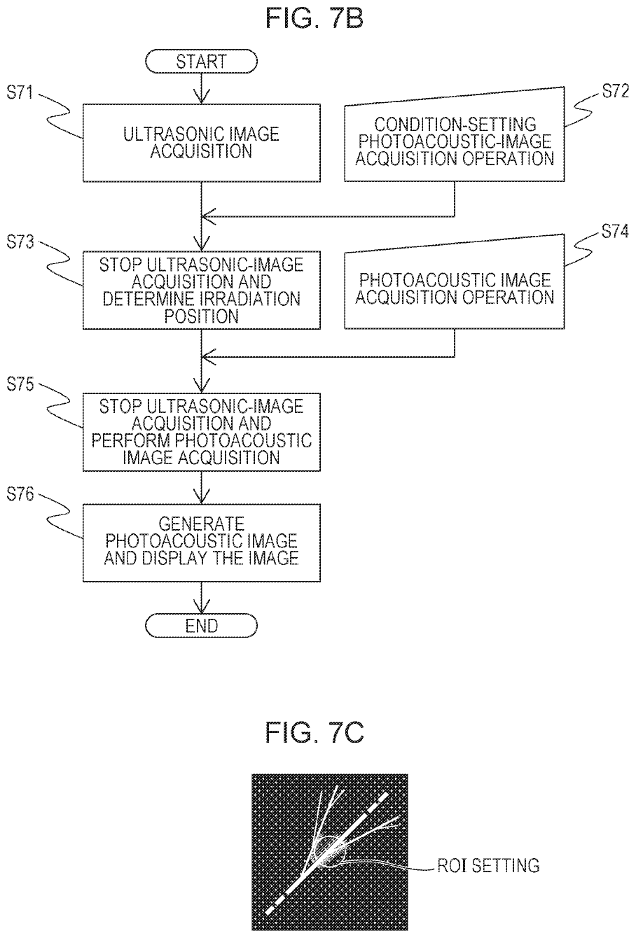

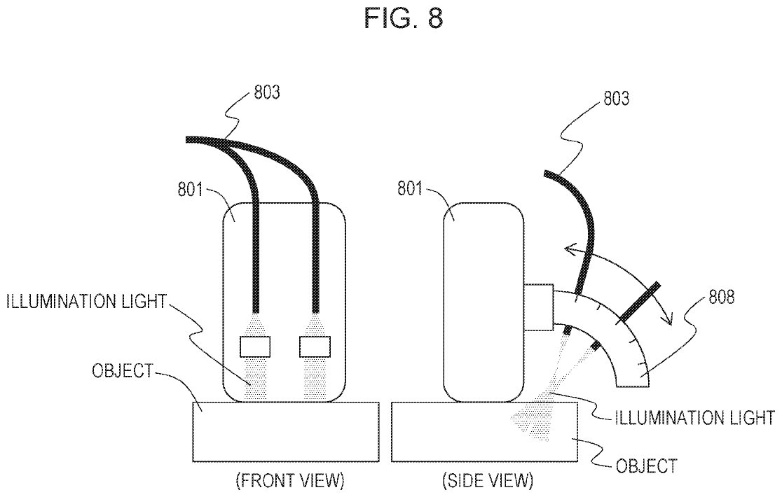

FIG. 7A illustrates a configuration in which a display unit 15 for presenting the irradiation position is added to the configuration in FIG. 1. The display unit 15 may be omitted, and the irradiation position information may be displayed on the monitor 4.

Referring to FIG. 7B, the process of determining the irradiation position includes the following steps.

Step 71 (S71) is an ultrasonic image acquisition step. A method for acquiring an ultrasonic image is the same as step 41 (S41) described with reference to FIG. 4A of the first embodiment, and a description here will be omitted.

Step 72 (S72) is a condition-setting photoacoustic-image acquisition operation. The operator operates the condition-setting image acquisition using the input unit 5 in FIG. 7A.

At step 73 (S73), the ultrasonic image acquisition is stopped according to the operation at S72, and an irradiation position at which the contrast of the ROI is high is acquired while the illumination light is moved within the movable range of the irradiation position using the varying unit 8. The irradiation position during the condition-setting image acquisition may be displayed on the display unit 15. This allows the operator to determine an irradiation position at which the contrast of the ROI is high while viewing the ROI in the object displayed on the monitor 4 and the irradiation position displayed on the display unit 15. A method for acquiring the photoacoustic image is the same as those at step 45 (S45) and step 46 (S46) described with reference to FIG. 4A of the first embodiment, and a description here will be omitted.

Step 74 (S74) is a photoacoustic image acquisition step, at which an irradiation position for improving the contrast of the ROI that is found out by the operator at S73 is set using the input unit 5.

At step 75 (S75), the ultrasonic image acquisition is stopped according to the operation at S74, and photoacoustic image acquisition is performed.

The details are the same as those of step 45 (S45) described with reference to FIG. 4A of the first embodiment, and a description thereof will be omitted.

Step 76 (S76) is an imaging step, at which the generated photoacoustic image is displayed on the monitor 4. The details are similar to those of step 46 (S46) described with reference to FIG. 4A in the first embodiment, so that a description thereof will be omitted.

The method described above allows a photoacoustic image under high contrast condition, which is highly visible for the operator, to be acquired by the condition-setting photoacoustic image acquisition.

### Fifth Embodiment

In the condition-setting method of the fourth embodiment, at S73 the operator determines an irradiation position at which the contrast of the ROI is high, and at S74 the operator sets a desired irradiation position. In contrast, in a condition-setting method of a fifth embodiment, the irradiation position is automatically set. A flowchart therefor is common to the flowchart in FIG. 7B, so that FIG. 7B in which “0” is added to each step number is used here.

Step 710 (S710) is an ultrasonic image acquisition step.

Step 720 (S720) is an ROI setting and condition-setting photoacoustic-image acquisition operation. The operator sets the region of interest using the input unit 5 while viewing the ultrasonic image displayed at S710. Thereafter, the operator operates condition-setting image acquisition using the input unit 5.

At step 730 (S730), the ultrasonic image acquisition is stopped according to the operation at S72, and a photoacoustic image is acquired while the position of the illumination light is moved using the varying unit 8. The information processing unit 2 determines an irradiation position where the contrast of the ROI is highest and causes the control unit 6 to move the illumination light to reach the irradiation position.

Step 740 (S740) is a photoacoustic image acquisition step, at which the operator performs a photoacoustic image acquisition operation using the input unit 5.

At step 750 (S750), the ultrasonic image acquisition is stopped according to the operation at S740, and photoacoustic image acquisition is performed.

Step 760 (S760) is an imaging step, at which the generated photoacoustic image is displayed on the monitor 4.

For the determination at S730 on the irradiation position where the contrast of the ROI is highest, the luminance of a photoacoustic image in the set ROI is determined. The contrast is calculated (the maximum value/the average value), and an irradiation position where the contrast is highest is determined. For example, FIG. 7C schematically illustrates part of the photoacoustic image in which the image acquisition target, noise, and artifacts are mixed. The luminance of the interior of the set ROI is determined for each of voxel (for 3D display), pixel (for 2D display), and 3D maximum intensity projection (MIP) with a predetermined thickness. The luminance is represented as 16 bits (65,536 levels of gray). The maximum luminance in the ROI is interpreted as the image acquisition target, and the average value is mainly interpreted as noise and an artifact. The contrast is determined from the ratio. The contrast is obtained from the ratio of the maximum value to the average value in the ROI but is not limited thereto. Alternatively, a method of isolating the image acquisition target from noise and artifacts more closely using an image recognition technique and determining the contrast from the ratio.

The method described above allows a photoacoustic image in which the contrast of the ROI is high to be acquired by the condition-setting photoacoustic image acquisition.

A varying unit that varies the irradiation position of the object is provided to change the irradiation position of light applied to the object according to the depth of the region of interest of the object. This improves the contrast of the photoacoustic image of the object.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-213423, filed Oct. 31, 2016, which is hereby incorporated by reference herein in its entirety.

1. An information acquisition apparatus comprising:
a light source configured to apply light to an object;
an ultrasonic probe configured to receive a photoacoustic wave generated from the object irradiated with the light and convert the photoacoustic wave to an electrical signal;
an information acquisition unit configured to acquire information on the object based on the electrical signal;
a receiving unit configured to receive an instruction on a condition for acquiring the information on the object;
a varying unit configured to vary an irradiation position of the light applied from the light source to the object; and
a control unit configured to control the varying unit,
wherein the control unit is configured to be capable of controlling the varying unit based on the instruction received by the receiving unit.

2. The information acquisition apparatus according to claim 1, further comprising an input unit configured to be capable of inputting the instruction.

3. The information acquisition apparatus according to claim 1, further comprising a display control unit configured to be capable of controlling display of image information on the object, the image information being acquired based on the information on the object.

4. The information acquisition apparatus according to claim 1, further comprising a display unit configured to be capable of displaying the image.

5. The information acquisition apparatus according to claim 1, wherein the varying unit comprises an actuator configured to vary at least part of an emission end of the light emitted from the light source.

6. The information acquisition apparatus according to claim 1,
wherein an acoustic matching material that allows light and a photoacoustic wave to pass through is disposed between the ultrasonic probe and the object, and
wherein a range in which an irradiation position of the light applied from the light source to the object is moved by the varying unit comprises a bright-field region and a dark-field region.

7. The information acquisition apparatus according to claim 1,
wherein the at least one emission end of the light emitted from the light source comprises a plurality of emission ends, and
wherein the varying unit is configured to be capable of switching among the emission ends to emit the light.

8. The information acquisition apparatus according to claim 1, wherein the range in which the irradiation position is moved by the varying unit is half or more of an irradiation region to which the light is applied.

9. The information acquisition apparatus according to claim 1, further comprising a photoacoustic probe, the photoacoustic probe comprising:
a casing containing the ultrasonic probe and an emission end of the light applied to the object, and
a covering unit covering a surface of the casing adjacent to the object.

10. The information acquisition apparatus according to claim 1, wherein the condition for acquiring the information on the object comprises information on a region of interest of the object.

11. The information acquisition apparatus according to claim 1,
wherein the condition for acquiring the information on the object comprises a depth of a region of interest of the object, and
wherein the control unit is configured to be capable of controlling the varying unit based on an expression or a table for determining an irradiation position according to the depth of the region of interest of the object received by the receiving unit.

12. The information acquisition apparatus according to claim 1, wherein the condition for acquiring the information on the object comprises the depth of the region of interest of the object, and
wherein the control unit is configured, when the region of interest is shallow, to control the irradiation position of the light from the light source to the object to come close to the ultrasonic probe, and when the region of interest is deep, to control the irradiation position of the light from the light source to the object to come away from the ultrasonic probe.

13. The information acquisition apparatus according to claim 1, wherein the condition for acquiring the information on the object comprises the depth of the region of interest of the object, and
wherein the control unit is configured to control the irradiation position of the light from the light source to the object to come away from the ultrasonic probe as the region of interest of the object becomes deeper.

14. The information acquisition apparatus according to claim 1, wherein the information acquisition unit is configured to calculate at least one of light amount distribution in the object, an absorption coefficient in the object, and oxygen saturation in the object according to the irradiation position of the light from the light source to the object.

15. The information acquisition apparatus according to claim 1, wherein the control unit is configured to control a total amount of the light applied to the object according to the irradiation position of the light from the light source to the object.

16. The information acquisition apparatus according to claim 1, wherein the information acquisition unit is configured to calculate light amount distribution in the object, an absorption coefficient in the object, and oxygen saturation in the object according to the irradiation position of the light from the light source to the object and the total amount of the light applied to the object.

17. The information acquisition apparatus according to claim 1, further comprising:
a display control unit configured to control display of the image acquired based on the information on the object,
wherein the control unit is configured to display the irradiation position of the light from the light source to the object, the irradiation position being changed by the varying unit.

18. The information acquisition apparatus according to claim 1, wherein the receiving unit receives the irradiation position of the light from the light source to the object.

19. A method for acquiring information, the method comprising the steps of:
receiving a photoacoustic wave generated from an object irradiated with light and converting the photoacoustic wave to an electrical signal;
acquiring information on the object based on the electrical signal;
receiving an instruction on a condition for acquiring the information on the object; and
controlling an irradiation position of the light applied to the object according to the instruction.

20. The method for acquiring information according to claim 19, wherein the control step comprises a step of changing the irradiation position of the light to the object according to a depth of a region of interest of the object when an instruction on the depth of the region of interest is received at the receiving step.

21. The method for acquiring information according to claim 19, further comprising the steps of:
performing condition-setting image acquisition for acquiring a photoacoustic image while controlling the irradiation position within a movable range of the irradiation position;
displaying the irradiation position during the condition-setting image acquisition on a display unit;
upon receiving an instruction on the irradiation position,
controlling the irradiation position according to the instruction; and
acquiring a photoacoustic image of the object at the irradiation position.

22. The method for acquiring information according to claim 19, further comprising the steps of:
upon setting a region of interest of the object and starting condition-setting image acquisition,
performing condition-setting image acquisition for acquiring the information on the object while controlling the irradiation position within a movable range of the irradiation position;
determining the irradiation position where the set region of interest has high contrast;
controlling the irradiation position according to the determination; and
acquiring a photoacoustic image of the object at the irradiation position.
